(19) United States
(12) Patent Application Publication
(10) Pub. No.: US 2017/0369525 A1
(43) Pub. Date: Dec. 28, 2017

(54) SITE-SPECIFIC CONJUGATION THROUGH GLYCOPROTEINS LINKAGE AND METHOD THEREOF

(71) Applicants: Development Center for Biotechnology, New Taipei City (TW); DCB-USA LLC, Wilmington, DE (US)

(72) Inventors: Chao-Pin Lee, New Taipei City (TW); Cheng-Chou Yu, New Taipei City (TW); Chi-Huey Wong, New Taipei City (TW); Chuan-Lung Hsu, New Taipei City (TW); Chun-Chung Lee, New Taipei City (TW); Shih-Hsien Chuang, New Taipei City (TW); Ta-Tung Yuan, New Taipei City (TW); Yi-Jen Chen, New Taipei City (TW); Yu-Chin Nieh, New Taipei City (TW)

(73) Assignees: Development Center for Biotechnology, New Taipei City (TW); DCB-USA LLC, Wilmington, DE (US)

(21) Appl. No.: 15/541,020

(22) PCT Filed: Dec. 31, 2015
(86) PCT No.: PCT/US2015/068300

§ 371 Date: Jun. 30, 2017

Related U.S. Application Data
(60) Provisional application No. 62/099,052, filed on Dec. 31, 2014.

(51) Int. Cl. C07K 1/107; C07K 1/08; A61K 39/395; C07K 16/18; A61K 47/68

(52) U.S. Cl. CPC C07K 1/1077; C07K 16/18; A61K 47/6889; A61K 39/395; C07K 1/08

(57) ABSTRACT

A method for specific linkage to a glycoprotein includes obtaining a glycoprotein having a monoglycan or diglycan attached thereto; producing a reactive functional group on a sugar unit on the glycoprotein; and coupling a linker or a payload to the reactive functional group on the glycoprotein.

### BACKGROUND OF INVENTION

Antibody-drug conjugates (ADCs) are one of the new methods for antibody modification to increase the potency and therapeutical window. ADCs are composed of an antibody and biological active cytotoxic payloads through a specific linker and designed as a targeted therapy for the treatment of cancer patients. Antibody Drug Conjugates are examples of bioconjugates and immunoconjugates.

Recently, research on novel binding ways between antibody and linkers attract lots of attention. The classical conjugation chemistries to prepare ADCs by targeting primary amines or hinge disulfides have a number of shortcomings including heterogeneous product profiles, linkage instability and active-site binding reduction. Novel site-specific conjugation method by targeting glycosylation site on antibodies may be a good approach.

Glycosylation is one of the most common post-translational modifications of proteins. Glycosylation affects the functions, immunogenicities, serum stabilities, and protease-resistance of proteins, including antibodies. The initial few sugar residues in N-glycosylation are well conserved and typically comprise an N-acetyl glucosamine attached to γ-amide of the Asn residue. This first sugar residue, N-acetyl glucosamine, is typically linked to a second N-acetyl glucosamine, which is in turned linked to a mannose.

All antibodies contain variable regions and constant domains. The variable regions recognize antigens directly. The molecular structures of the constant domains vary between different types of antibodies. Upon binding to an antigen, an antibody may interact with receptors on different immune cells. Different glycosylation patterns in the Fc regions will affect not only the efficacies but also the stabilities of antibodies. The glycosylation patterns in the Fc regions can be modified to change the functions of an antibody. Glycan engineering can be used to modulate the glycan structures on the Fc of an antibody, its affinity to the receptor, and its immune responses.

To improve the efficacies and glycan structure consistency of therapeutic antibodies, there have been attempts to developed glyco-engineering platforms for the production of homogeneously glycosylated antibodies, see e.g., U.S. Patent application publication No. 2011/0263828 A1; PCT publication WO 2007/146847 A2 and U.S. Patent provisional application No. 61/986,471. These patents illustrated several different methods to generate antibodies with only one sugar (1) treating an antibody with endo-β-N-acetylglucosaminidase, such as endo-S followed by α-fucosidase under different conditions to remove most of the glycans and leave only a single sugar unit (i.e., GlcNAc); (2) employing specifically engineered cells to produce homogeneous mono-GlcNAc antibodies. These approaches may yield antibodies having one or two sugar units (GlcNAc, GlcNAc-Fuc and GlcNAc-Gal).

### SUMMARY OF INVENTION

Embodiments of the invention relate to methods for producing antibody-drug conjugates (ADCs) by conjugation with the N-linked mono-GlcNAc sites, or N-linked GlcNAc-Gal sites, or N-linked GlcNA-Fuc sites on antibodies. For antibodies, the N-linked glycans may be attached to a constant domain, such as N-linked GlcNAc attached to Asn297 of IgG1 constant domains. Methods of the invention use either enzymatic or chemical methods to produce covalent bonding between antibodies and linkers (or other moieties such as a therapeutic agent). These homogeneous mono-GlcNAc antibodies can be generated from methods know in the art and can be used as is or can be used for further modifications, as illustrated in FIG. 2.

One aspect of the invention relates to methods for specific linkage to glycoproteins. A method in accordance with one embodiment of the invention may include obtaining a glycoprotein having a monoglycan or diglycan attached thereto; producing a reactive functional group on a sugar unit on the glycoprotein; and coupling a linker or a payload to the reactive functional group on the glycoprotein.

In accordance with any of the above embodiments of the invention, the glycoprotein may be any glycoprotein, such as one selected from the group consisting of mono-glycan(GlcNAc), diglycan (GlcNAc-Fuc), and diglycan (GlcNAc-Gal).

In accordance with any of the above embodiments of the invention, the liner may comprise a functional group for coupling to an aldehyde group, such as a hydrazide moiety, a hydrazino-Pictet-Spengler ligation moiety, an amine moiety, an oxazoline moiety, a methylhydrazine, and an N-methyl hydroxylamine.

In accordance with any of the above embodiments of the invention, a method to produce a reactive function group on the glycoprotein may involve a chemical reaction, such as oxidation by a periodate, or an enzymatic reaction, such as oxidation by galactoside oxidase.

In accordance with any of the above embodiments of the invention, the ADCs may have a payload directly conjugated with the glycoprotein. Alternatively, the ADCs may have a payload conjugated via a linker to the glycoprotein.

In accordance with any of the above embodiments of the invention, the payload is a therapeutic agent, a cytotoxic agent, or an imaging agent.

### BRIEF DESCRIPTION OF THE DRAWINGS

FIG. 1 shows two exemplary methods for generating a monoglycan glycoprotein. As shown in method 1 (Panel A), one may generate glycoproteins having one or two sugar units attached to the glycoproteins by using a endoglycosidase, such as Endo S. Even if the initial products may contain a mixture of a non- and di-glycan, it is possible to further clean up the reaction products with a second enzyme, fucosidase, to trim the second glycan unit. Method 2 (Panel B) shows an alternative method, in which a native glycoprotein may be produced and then trimmed with suitable glycosidases. For example, it may be treated with neuraminidase, followed by β-galactosidase.

FIG. 2 shows schematics for producing various mono- or di-glycan glycoproteins, using herceptin antibody as an example, in accordance with embodiments of the invention. As shown, a monglycan herceptin antibody having only GlcNAc may be produced by Endo S treatment, followed with fucosidase treatment. Alternatively, this may be produced with neuraminidase treatment, followed with galactosidase treatment, of a herceptin antibody expressed from a cell harboring Endo S. Herceptin antibody with a diglycan, GlcNAc-Fuc, may be produced with Endo S treatment or simply by expressing the antibody in a cell harboring Endo S (or a similar endoglycosidase). A diglycan, GlcNAc-Gal, analog of herceptin antibody may be produced with an antibody expressed in a cell harboring Endo S, followed by treatment with neuraminidase.

FIG. 3 shows schematics illustrating various methods for producing ADCs from the various forms of glycoproteins having one or two glycan units. First, the glycan unit is oxidized with an enzyme (e.g., galactose oxidase or a similar sugar oxidase) or by chemical reaction (e.g., periodate oxidation) to generate a reactive group (e.g., an aldehyde) for coupling or a linker or a payload directly.

FIG. 4 shows various ADCs prepared in accordance with embodiments of the invention.

FIG. 5 shows binding of the various ADCs of FIG. 4, confirming that conjugation of the payload or a linker-payload to a glycoprotein, in accordance with embodiments of the invention, does not damage the glycoprotein.

FIG. 6 shows binding of the various ADCs of FIG. 4, confirming successful conjugation of the payload or a linker-payload.

FIG. 7 shows a schematic illustrating a method of using periodate to generate a reactive group for coupling with a linker and payload in accordance with embodiments of the invention.

FIG. 8 shows various ADCs as examples produced with a method of using periodate to generate a reactive group for coupling with a linker and payload, as illustrated in FIG. 7, in accordance with embodiments of the invention.

FIG. 9 shows herceptin bindings of the ADCs of FIG. 8, illustrating that the periodate reaction does not damage the antibody.

FIG. 10 shows avoiding bindings of the ADCs of FIG. 8, illustrating successful conjugation of a payload (i.e., biotin).

FIG. 11 shows a schematic illustrating conjugation of a payload to a diglycan antibody, which coupling may occur on the first, second, or both sugar units. Galatosidase may be used to cleave between the first and second sugar units to assess the coupling sites.

FIG. 12 shows biotin-avidin binding of the ADCs illustrated in FIG. 11. Galatosidase treatment reduces some binding, indicating that at least some conjugation occurred on the galactose unit.

FIG. 13 shows a schematic illustrating conjugation of a payload to glycoprotein may be effected with different reactive linkage groups after periodate reaction, such as using a hydrazine function group on a linker or an amino group in a Schiff base formation (including Amidori reaction) or in a Pictet-Spengler reaction.

FIG. 14 shows ADC produced with a hydrazine function group as illustrated in FIG. 13.

FIG. 15 shows avidin bindings of the ADC of FIG. 14, illustrating successful conjugation of the payload to the antibody.

FIG. 16 shows various ADCs having a fluorescent group as a payload in accordance with embodiments of the invention.

FIG. 17 shows detection of the ADCs of FIG. 16. The left panel is an SDS-PAGE gel visualized with Coomassie Blue, showing the locations of the ADCs. The right panel shows a fluorograph, illustrating the successful conjugation of the fluorophores.

FIG. 18 shows various ADCs having a cytotoxic group as a payload in accordance with embodiments of the invention.

FIG. 19 shows cytotoxicity of the ADCs of FIG. 18, assed with two different cell lines, SK-BR-3 and MDA-MB-231. The results show that the ADCs are effective cytotoxic agents.

FIG. 20 shows schematics illustrating ADC conjugations using enzymatic oxidation to generate a reactive group on a sugar unit for conjugations, in accordance with embodiments of the invention.

FIG. 21 shows a reaction on galactose by galactose oxidase.

FIG. 22 shows various ADCs generated according to the schematic of FIG. 20.

FIG. 23 shows the avidin-biotin binding of the ADCs of FIG. 22, illustrating successful conjugation of the payload using the scheme of FIG. 20.

FIG. 24 shows ADCs generated at two different temperatures.

FIG. 25 shows avidin-biotin bindings of the ADCs of FIG. 25, illustrating successful conjugation of the payload at both temperatures.

FIG. 26 shows ADCs generation from glycoproteins having different sugar units, illustrating that it is possible to differentially conjugate one type of glycoprotein more readily than another type of glycoprotein by taking advantage of selectivity of an enzymatic reaction.

FIG. 27 shows avidin-biotin bindings of the ADCs of FIG. 26, illustrating successful selection of conjugation to one type of glycoprotein in accordance with embodiments of the invention.

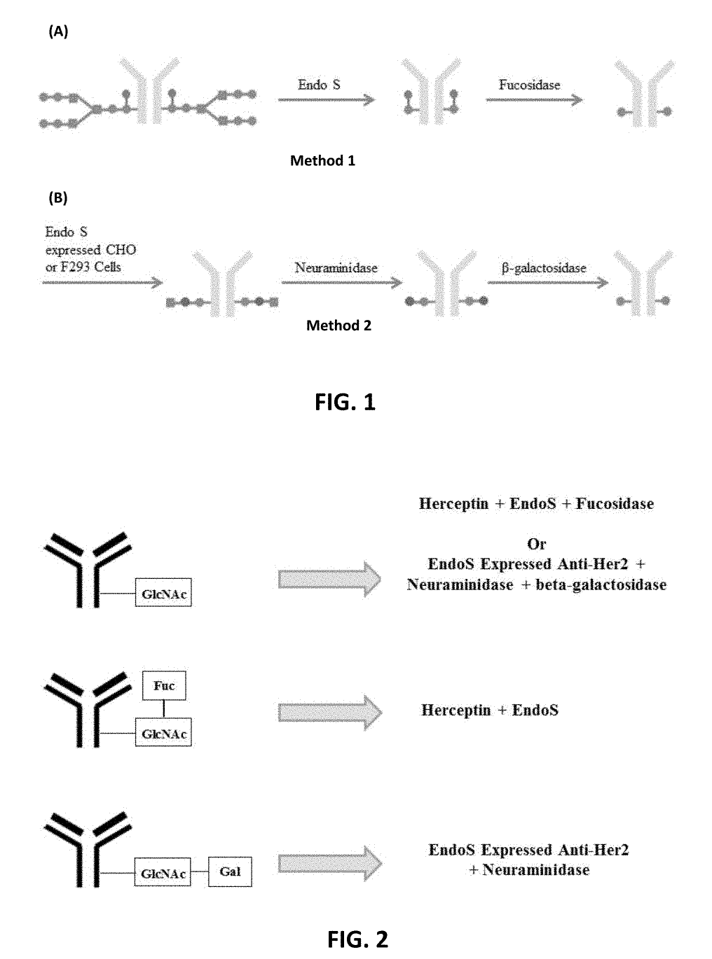

### DETAILED DESCRIPTION

Embodiments of the invention relate to methods for producing antibody-drug conjugates (ADCs) by conjugation with the N-linked mono-GlcNAc sites, or N-linked GlcNAc-Gal sites, or N-linked GlcNA-Fuc sites on antibodies. For antibodies, the N-linked glycans may be attached to a constant domain, such as N-linked GlcNAc attached to Asn297 of IgG1 constant domains. Methods of the invention use either enzymatic or chemical methods to produce covalent bonding between antibodies and linkers (or other moieties such as a therapeutic agent). These homogeneous mono-GlcNAc antibodies can be generated from methods know in the art and can be used as is or can be used for further modifications, as illustrated in FIG. 2.

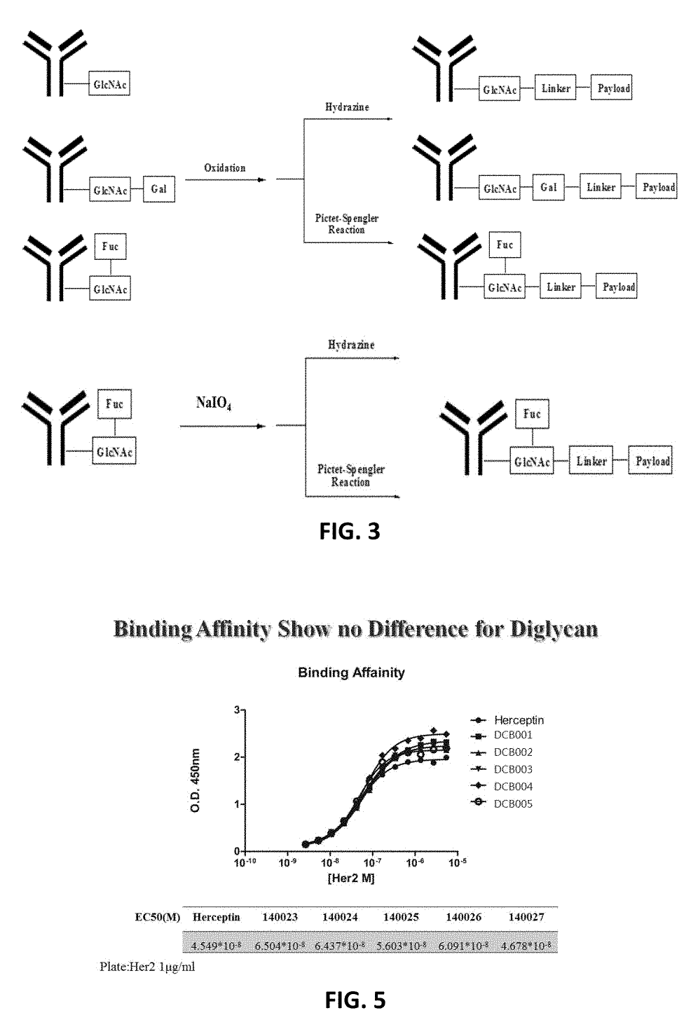

The mono- or di-glycan antibodies oxidized by either chemical methods, such as sodium periodate (NaIO₄), or enzymatic methods, such as galactose oxidase, can generate aldehydes on glycan moieties. Using hydrazine-containing linkers, one may react the linkers with the aldehyde group to afford site-specific conjugations (see FIG. 3). In addition, using other known procedures, including reductive amination, Pictet-Spengler reaction, or oxime formation, linkers may be attached to the mono- or di-glycan antibodies through these aldehyde tag sites (see FIG. 3).

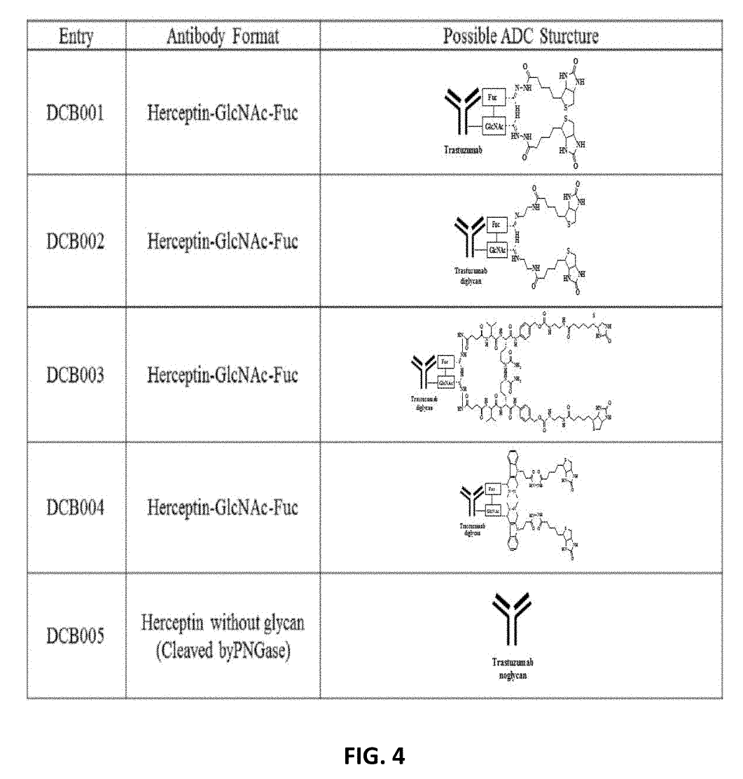

Using herceptin antibodies as an example, several ADC structures have been prepared using the methods illustrated in FIG. 3. The herceptin antibodies (Trastuzumab) having diglycans attached, i.e., GlcNAc-Fuc, are used for the examples. These compounds are shown in FIG. 4. In these examples, herceptin antibodies are conjugated to biotins, with or without a linker, to test the concept.

The binding activities of these conjugates were examined using both herceptin protein and avidin. As shown in FIG. 5, the binding affinities of these conjugates for herceptin do not show significant differences.

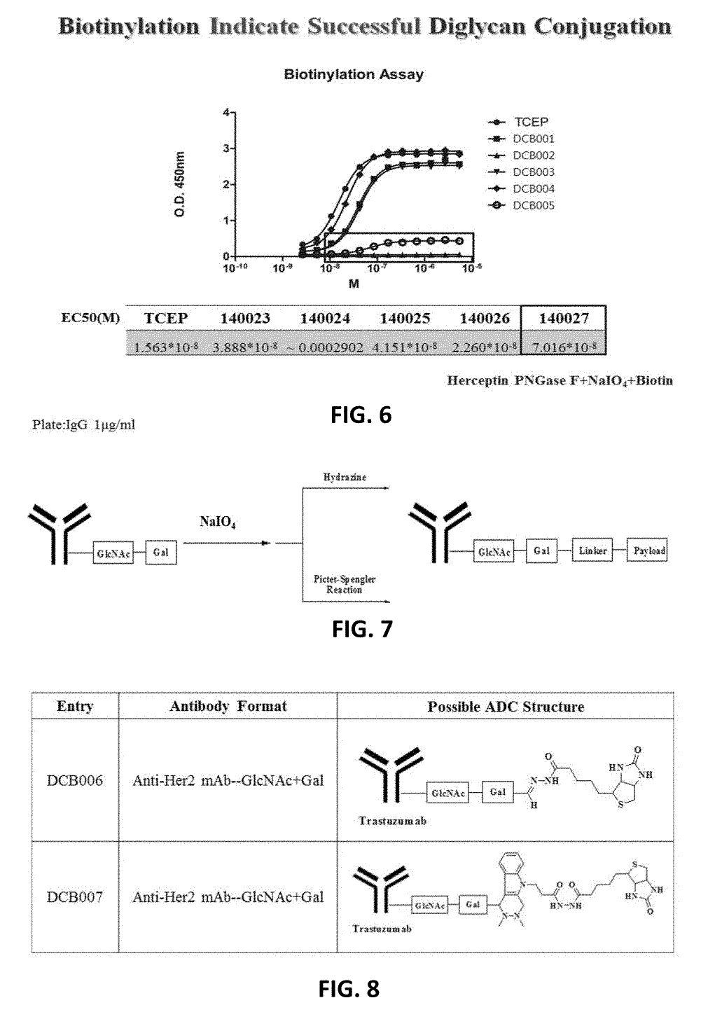

FIG. 6 shows the avidin bindings of these conjugates. DCB005 is a negative control, because the diglycan on herceptin antibody had been removed and there was no sites for biotin attachment. As shown in FIG. 6, most conjugates do show significant avoiding binding. These results prove that methods shown in FIG. 3 can be successfully used to make desired ADCs.

In addition to direct conjugation, the drug conjugate may be linked to an antibody via a linker. As illustrated in the scheme in FIG. 7, after oxidation of the glycan moiety to create aldehyde functional groups, a linker may be directly conjugate to the newly created aldehyde groups, then a payload (e.g., a therapeutic drug or a marker or an imaging agent) may be conjugated to the linker. Alternatively, the linker and the payload may be first coupled, and then the linker-payload is conjugated with the antibody.

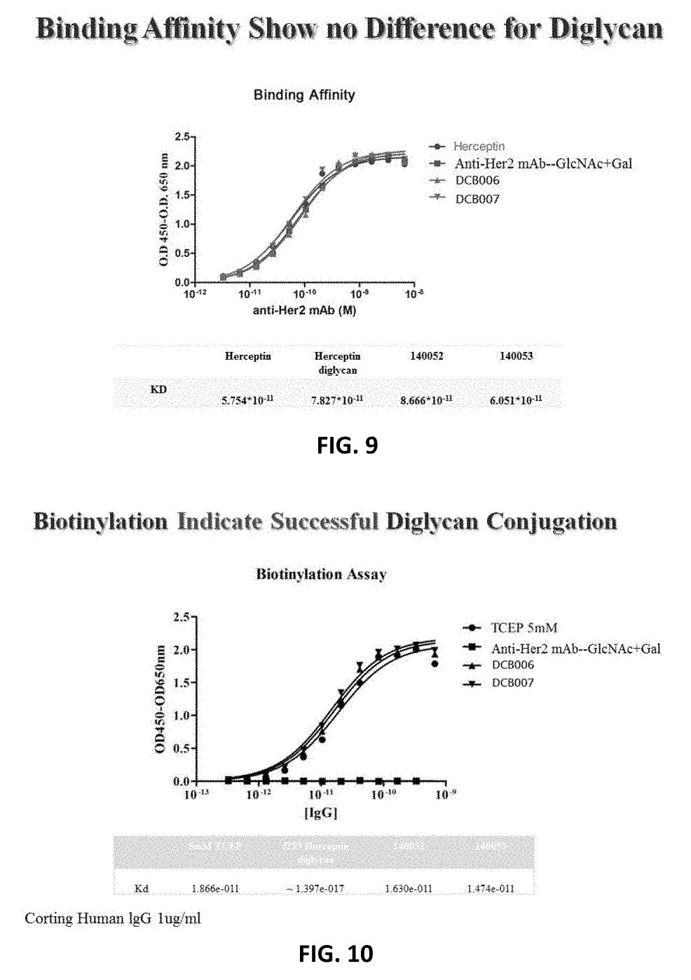

FIG. 8 shows an example of an ADC having a linker, as well as a control ADC without the linker. The bindings of these ADCs to herceptin and avidin were assessed. As shown in FIG. 9, there is no difference in the bindings of these ADCs to herceptin. FIG. 10 shows that the bindings of these ADCs to avidins are almost indistinguishable. These results indicate that whether a linker is used would not impact the binding of the antibody or the payload. Therefore, embodiments of the invention can be used to produce ADCs, with or without linkers.

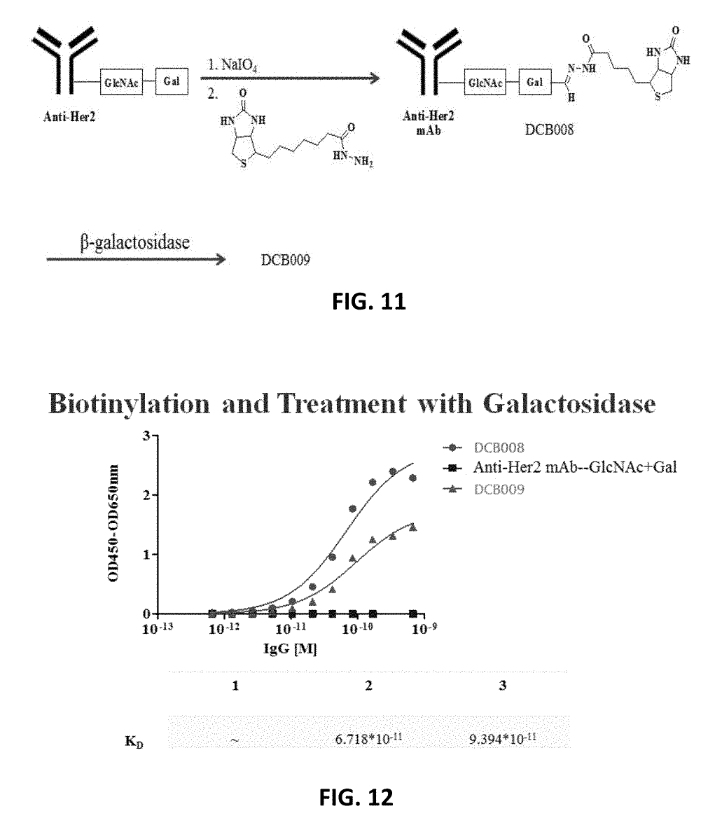

Periodate reactions can occur with any vixinyl alcohols. Therefore, the attachments of biotins to herceptin antibodies in the above examples can be on either sugar ring. As illustrated in FIG. 11, herceptin antibody contains a diglycan, GlcNAc-Gal. An ADC prepared from this antibody may have a payload (e.g., a biotin) attached to Gal, or GlcNAc, or both. One may use β-galactosidase to remove the Gal residue from ADC. If the payload is attached to Gal, it would be removed. On the other hand, if the payload is attached to GlcNAc or both, all or some payload may remain after β-galactosidase treatments.

As shown in FIG. 12, avidin binding is partially decreased after treatment with β-galactosidase. This result indicates that at least part of biotin is attached to the Gal unit.

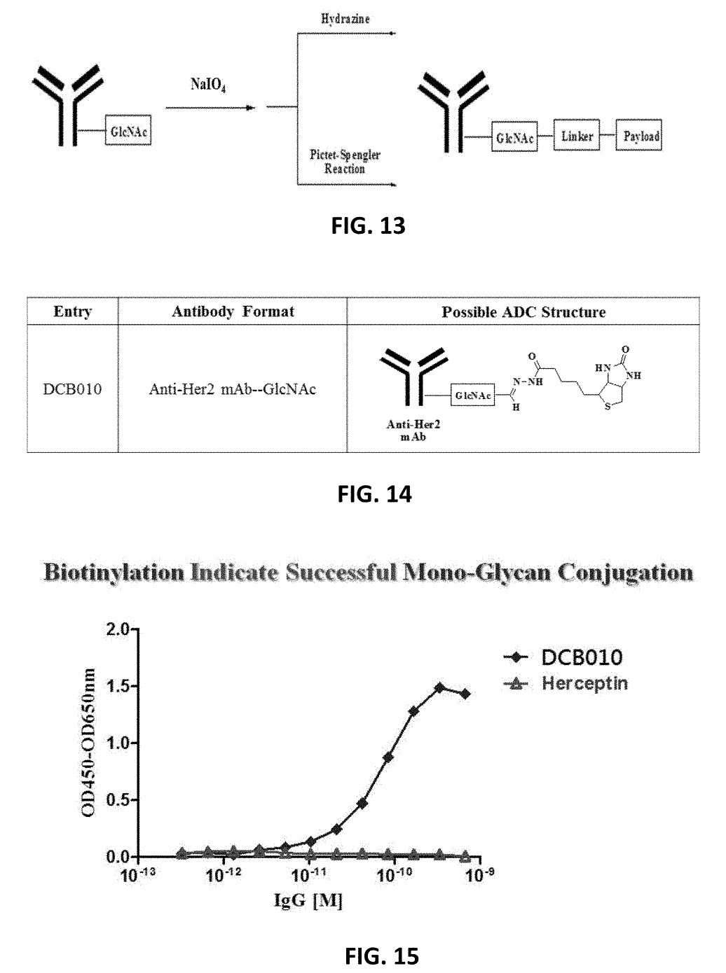

Embodiments of the invention may also use glycoproteins or antibodies having a monglycan attached thereto. As illustrated in FIG. 13, similar processes may be used to attach payloads on these antibodies or glycoproteins. An example of such ADCs is shown in FIG. 14.

FIG. 15 shows avidin binding by this ADC (DCB010). The results of this binding indicate that the ADC was successfully prepared with a mon-glycan antibody (or glycoprotein).

In accordance with embodiments of the invention, an ADC may comprise a glycoprotein or an antibody conjugated to a payload, with or without a linker therebetween. A glycoprotein or antibody preferably has one or two sugar units (i.e., mono glycan or diglycan) attached.

In accordance with embodiments of the invention, a payload may be a therapeutic agent, a cytotoxic agent, an imaging agent (which may be a fluorescence moiety, a radioactive nuclide, or an agent containing an NMR-reactive atom (e.g., ²H, ¹³C, ¹⁹F, ³¹P, etc.). Any therapeutic or cytotoxic agent may be used with an embodiment of the invention, such as anticancer agents (e.g., vincristine, taxol, gemcitabine, cisplatin, etc.). One skilled in the art would appreciate any therapeutic agents that are commonly used in ADCs may be used with embodiments of the invention.

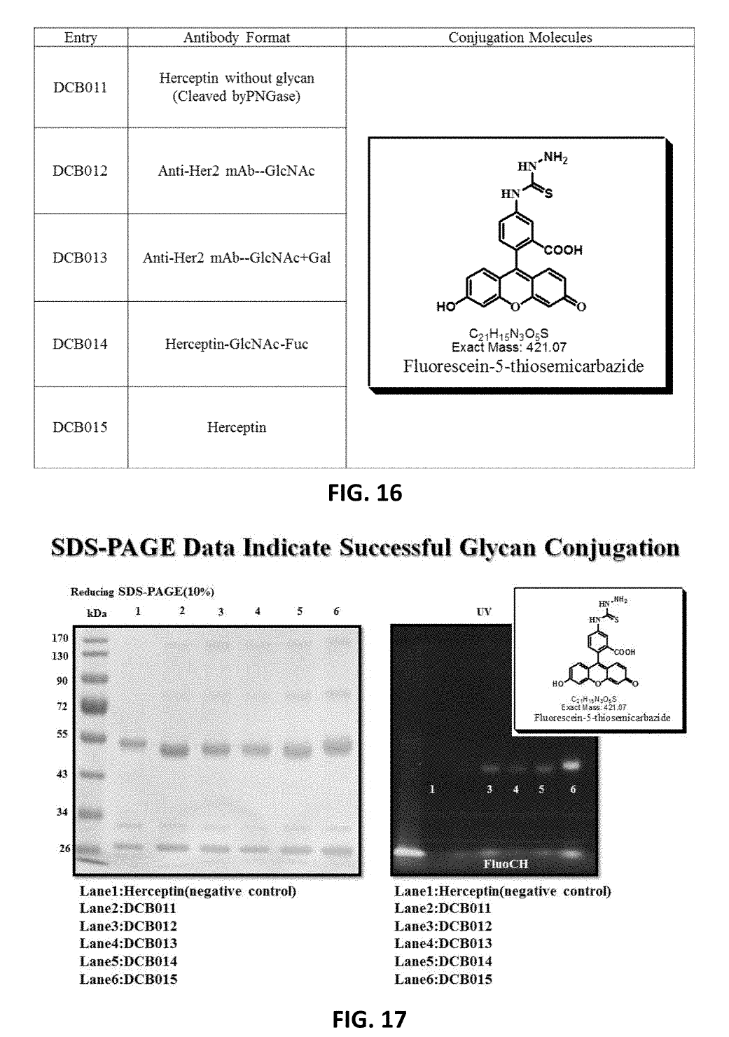

FIG. 16 shows some examples of ADCs in accordance with embodiments of the invention that contain an imaging agent. In these examples, fluorescein-5-thiosemicarbazide is used to couple to a glycoprotein or an antibody, with or without a linker. Such a fluorescent moiety can be used in imaging.

FIG. 17 shows an example of detection of the ADCs shown in FIG. 16. The left panel in FIG. 17 shows an SDS-PAGE gel probed with Coomassie Blue to locate the bands of the ADCs. The right panel in FIG. 17 shows a fluorograph, showing that ADCs contain the fluorescent moiety. While this example illustrates a fluorescence detection, one skilled in the art would appreciate that other imagining methods may also be used, such as radiograph (using a radioactive moiety in the payload), spectral or fluorescent imaging (e.g., NIR dyes, fluorophores in the payload), PET (positron emission tomography, e.g., ¹⁸F), or SPECT (single photon emission computed tomography). Any suitable radionuclides, such as Cu-64, Ga-68, F-18, Tc-99, Lu-177, Zo-89, Th-227 and Gd-157, may be used with embodiments of the invention.

In accordance with embodiments of the invention, a linker may be used to connect a glycoprotein or antibody to a payload. The linker may help in efficient delivery and/or effective release of payload once the ADC is delivered to the target site (e.g., in the cancer cells).

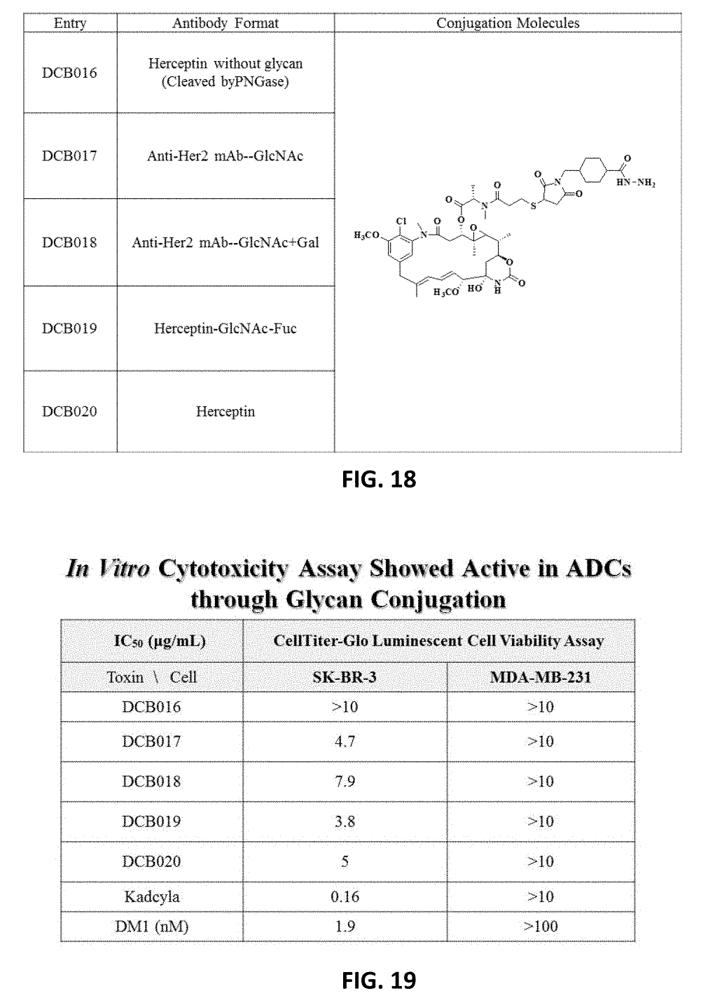

Some embodiments of the invention may have payloads that can kill the target cells. FIG. 18 shows examples of ADCs containing a cytotoxic agent as a payload. FIG. 19 shows results (IC₅₀) of these ADCs with SK-BR-3 cells and MDA-MB-231 cells. As shown in FIG. 19, ADCs in accordance with embodiments of the invention can be effective in cell killing or growth inhibition.

For the conjugation of a payload to a glycoprotein or an antibody, embodiments of the invention takes advantages of the glycan moiety. The sugar unit may be oxidized to generate a reactive group (e.g., an aldehyde group) for coupling with a payload or a linker. Common methods for generating a reactive group from a sugar unit may include oxidation with periodate (e.g., NaIO₄), as illustrated in the above examples. In addition, enzymatic oxidation may also be used with embodiments of the invention.

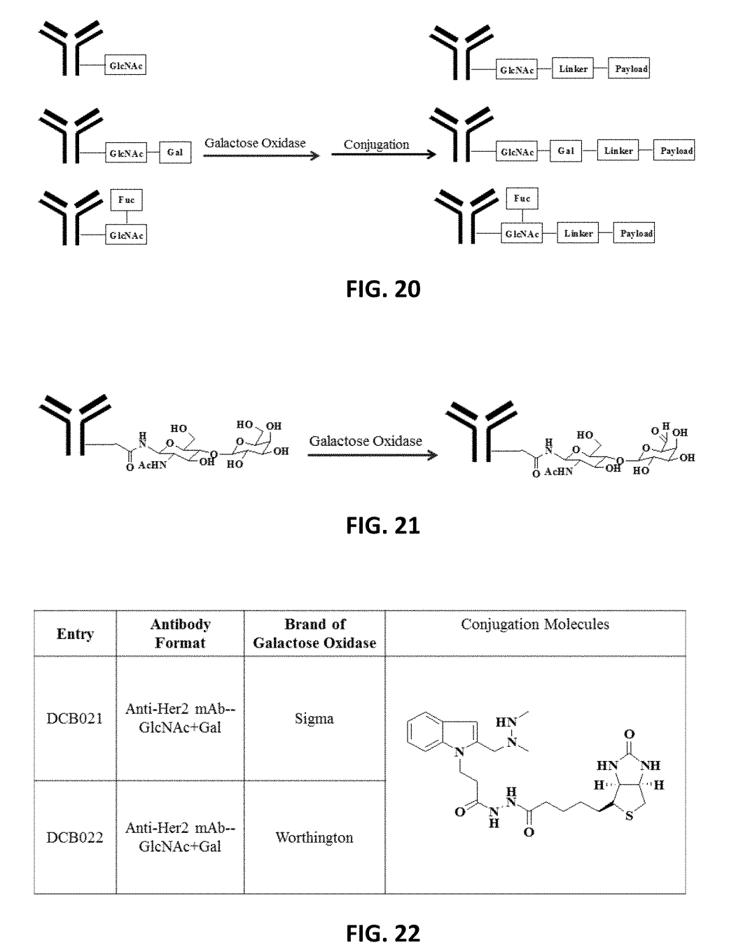

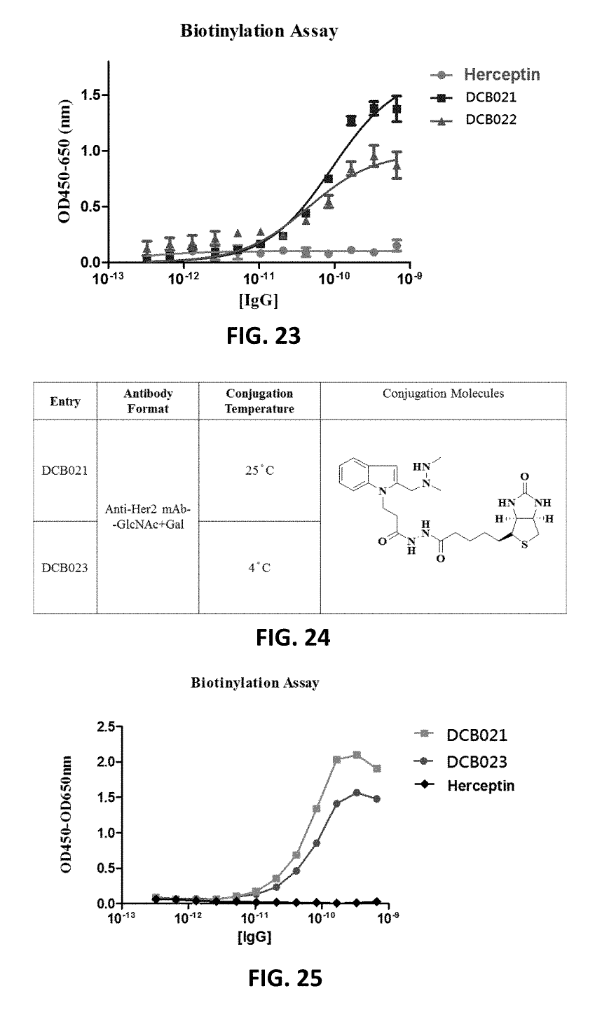

FIG. 20 shows a schematic illustrating a method using galactose oxidase to produce ADCs in accordance with embodiments of the invention. FIG. 21 shows the chemical reaction catalyzed by galactose oxidase. FIG. 22 shows some examples of ADCs produced by galactose oxidase. FIG. 23 shows avidin-biotin bindings of the two ADCs generated using galactose oxidase. The results show that both galactose oxidases are effective, though one is better than the other.

FIG. 24 shows two ADCs generated at two different temperatures, 25° C. and 4° C. The conjugation reactions went well at both temperatures, as illustrated in the biotin-avidin binding assay (FIG. 25). These results indicate that the conjugation reactions in accordance with embodiments of the invention are tolerant of a wide range of reaction conditions.

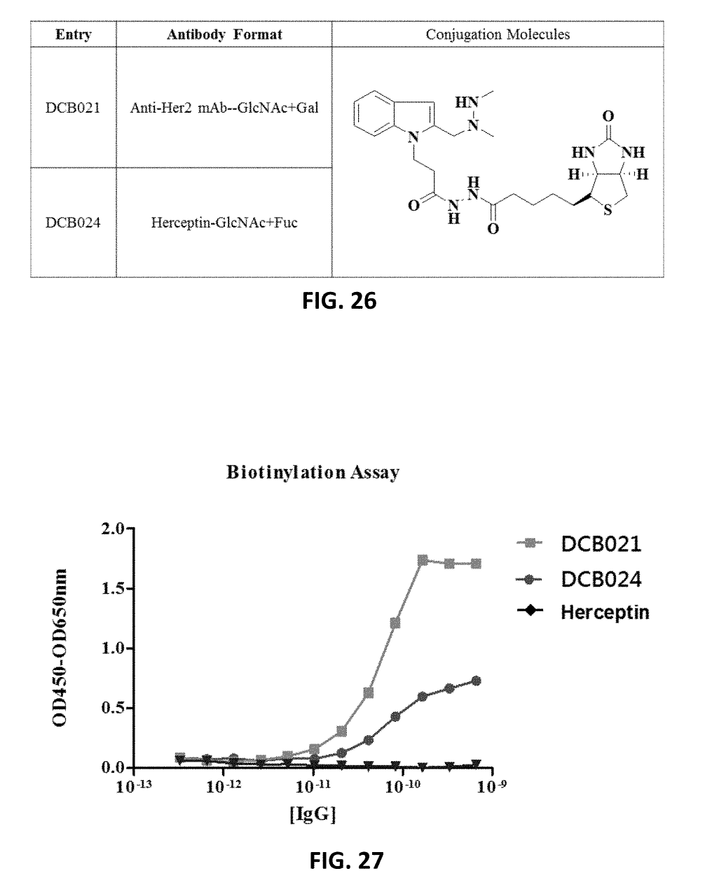

In accordance with embodiments of the invention, when a glycoprotein or an antibody contain different sugar units, it is possible to selective form ADC with one type of sugar more preferably than the other sugar units. FIG. 26 shows an example of herceptin antibodies containing different diglycans, one with GlcNAc-Gal and the other with GlcNAc-Fuc. By using an enzyme that prefers one sugar over the other, it would be possible to generate an ADC more preferably in one type of glycoprotein than the other.

As shown in the avidin-biotin assays, using a galactose oxidase to generate a reactive group for conjugation proceeds more readily with a galactose unit than with a fucose unit. As a result, the ADC with herceptin-GlcNAc-Gal has a better coupling efficiency, than herceptin-GlcNAc-Fuc.

Embodiments of the invention takes advantage of glycoproteins or antibodies having one or two sugar units attached thereto to produce ADCs that are more homogeneous. One skilled in the art would appreciate that biologics with homogeneous compositions are important because they may be more effective, more stable, show better pharmacokinetic parameters, have a better defined property to facilitate formulation, etc.

The above examples illustrate the benefits of embodiments of the invention. The following specific examples will further illustrate these and other embodiments of the invention. One skilled in the art would appreciate that these examples are for illustration only and are not meant to limit the scope of the invention. One skilled in the art would appreciate that other modifications or variations of these examples are possible without departing from the scope of the invention.

### EXAMPLES

The following examples are presented to illustrated certain embodiments of the present invention, but should not be construed as limiting the scope of this invention. Unless otherwise indicated, each 1H NMR was data were obtained at 500 MHz. The abbreviations used herein are as follows, unless specified otherwise

Bu: butyl; Bn benzyl; BOC t-butyloxycarbonyl; BOP: benzotriazol-1-yloxy tri/dimethylamino-phosphonium hexafluorophosphate; HIPS: Hydrazino-Pictet-Spengler Ligation; DCC dicyclohexylcarbodiimide; DMF N,N-dimethylformamide; DMAP: 4-dimethylaminopyridine; EDC 1-(3-dimethylaminopropyl) 3-ethylcarbodiimide hydrochloride; EtOAc: ethyl acetate; Eq.: equivalent(s); HOBt hydroxybenztriazole; LAH: lithium aluminum hydride; MeOH: methanol; MHz: megahertz; MS(ES): mass spectrophotometer-electron spray; NMP N-methylpyrrolidinone; Ph: phenyl; Pr: propyl; TEA: triethylamine; THF: tetrandrofuran; TLC: thin layer chromatography; Tetrakis tetrakis(triphenylphosphine)palladium.

A “linker” is a molecule with two reactive termini, one for conjugation to an antibody (or glycoprotein) and the other for conjugation to a payload (e.g., a cytotoxin, a therapeutic agent, an imaging moiety (e.g., a fluorophore or a radioactive nuclide), etc.). The antibody conjugation reactive terminus of a linker is traditionally a site that is capable of conjugation to the antibody through a cysteine thiol or lysine amine group on the antibody, and so is typically a thiol-reactive group such as a double bond (as in maleimide) or a leaving group such as a chloro, bromo, or iodo, or an R-sulfanyl group, or an amine-reactive group such as a carboxyl group; besides diglycan moiety may attach to glycoproteins comprising mono-N-acetyl glucosamine so that is a new method to be a reactive terminus. In accordance with embodiments of the invention, a linker conjugates with a functional group that is derived from oxidation of a glycan. Such function group may be an aldehyde, and therefore the reactive group on a linker would be an amino group to form a Schiff base or a rearranged Schiff base linkage (e.g., an Amidori reaction).

The antibody conjugation reactive terminus of a linker is typically a site that is capable of conjugation to the cytotoxin through formation of an amide/ester bond with a basic amine/alcohol or carboxyl group on the cytotoxin, and so is typically a carboxyl, an alcohol, or a basic amine group. When the term “linker” is used in describing the linker in conjugated form, one or both of the reactive termini will be absent (such as the leaving group of the thiol-reactive group) or incomplete (such as the being only the carbonyl of the carboxylic acid) because of the formation of the bonds between the linker and/or the cytotoxin.

Glycoproteins are proteins that contain oligosaccharide chains (glycans) covalently attached to polypeptide side-chains. The carbohydrate is attached to the protein in a cotranslational or posttranslational modification. This process is known as glycosylation. Secreted extracellular proteins are often glycosylated. In proteins that have segments extending extracellularly, the extracellular segments are also glycosylated. Glycoproteins are often important integral membrane proteins, where they play a role in cell-cell interactions.

### Example 1

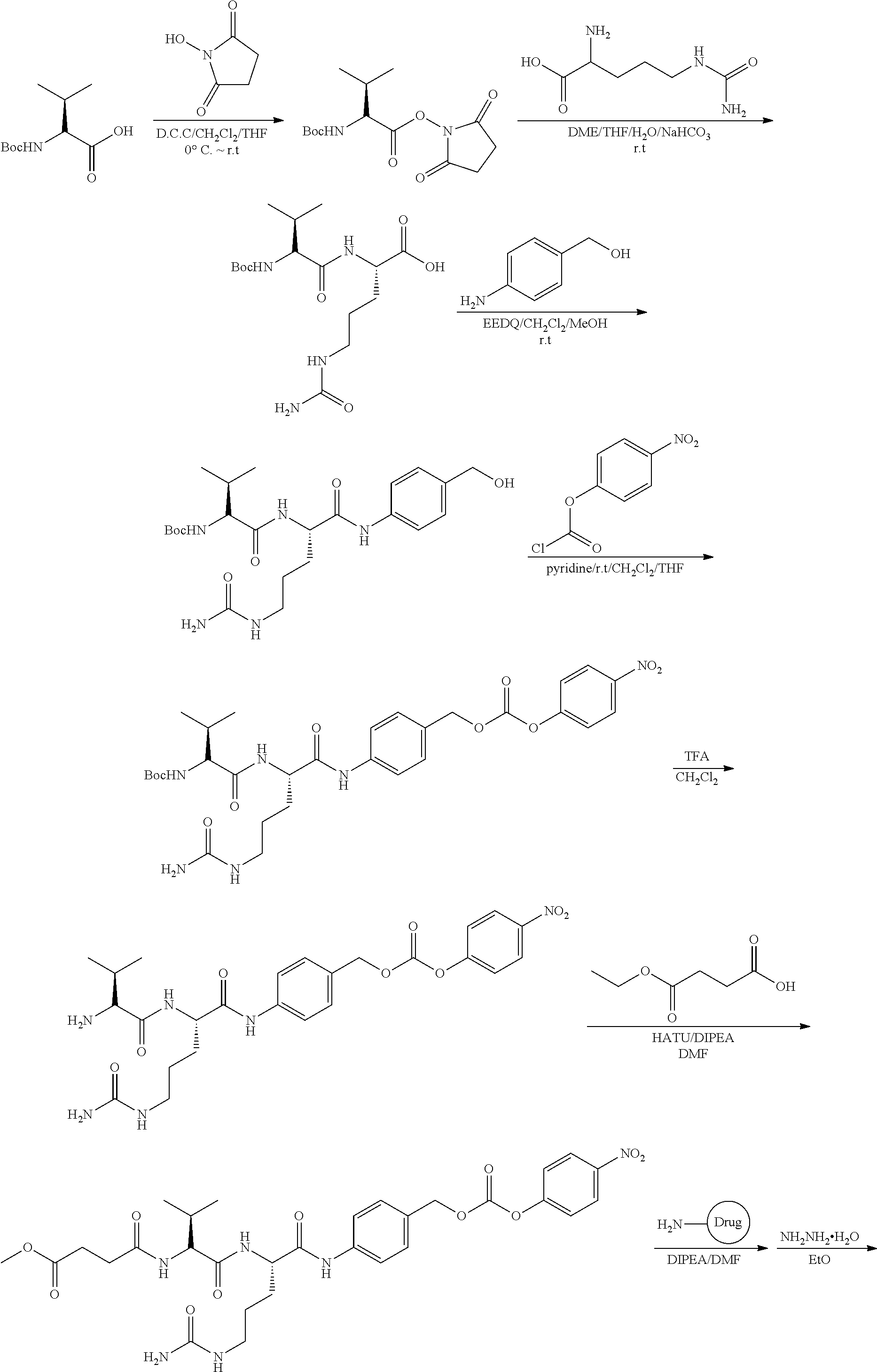

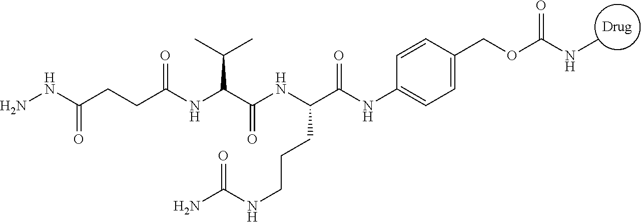

Boc-Val-OSu

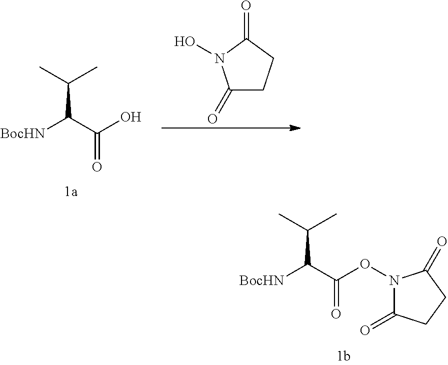

A solution of N-Hydroxysuccinimide (5.0 g, 43.44 mmol) and Boc-Val-OH (1a, 9.462 g, 43.44 mmol) in THF (83 mL) was stirred in room temperature for 3.0 minutes. Then DCC (9.856 g, 45.57 mmol) in CH₂Cl₂ (83 mL) was slowly added to the solution at 0° C. and then warmed to room temperature. The reaction mixture was stirred for further 18 hours. The solution was cooled to 0° C. and the precipitate was filtered and washed with EA (100 mL), dried over reduced pressure to give Boc-Val-OSu (1b, 13.28 g). ¹H NMR (500 MHz, DMSO-d6): δ: 5.02 (d, 1H), 4.60 (d, 1H), 2.85 (s, 4H), 2.31 (m, 1H), 1.48 (m, 9H), 1.05 (m, 6H). MS (M+1): 314.8.

Boc-Val-Cit

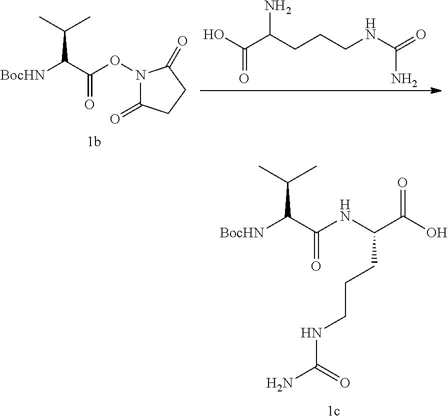

To a solution of NaHCO₃ (3.544 g, 42.20 mmol) in H₂O (100 mL) was added L-(+)-Citrulline (7.389 g, 42.2 mmol) in THF (25 mL) in room temperature. Then Boc-Val-OSu (1b, 12.62 g, 40.19 mmol) in DME (100 mL) was then slowly added into the solution. The reaction mixture was stirred at room temperature for 18 hours. Remove the organic solvent, add 10% citric acid to the solution and then extracted with 10% IPA/EA. The organic layer was washed with brine, dried over MgSO4(s), and concentrated under reduced pressure to provide Boc-Val-Cit (1c). ¹H NMR (500 MHz, DMSO-d6): δ: 4.37-4.41 (m, 1H), 3.90 (d, 1H), 3.12 (t, 2H), 1.51-20.6 (m, 5H), 1.44 (s, 9H), 0.97 (d, 3H), 0.92 (d, 3H). MS (M+1): 375.0.

Boc-Val-Cit-PABOH

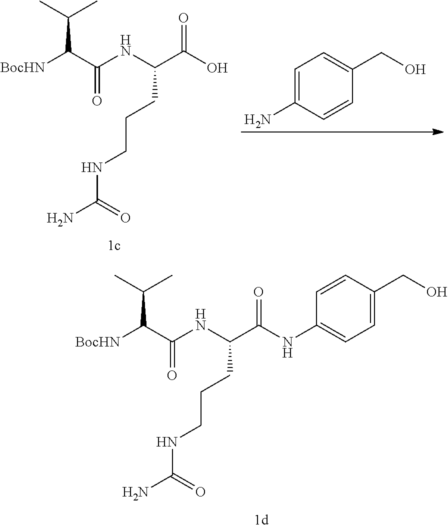

Boc-Val-Citrulline (1c, 12.93 g, 34.57 mmol) and 4-Aminobenzyl alcohol (PABOH, 4.683 g, 38.03 mmol) in CH₂Cl₂ (250 mL) and MeOH (125 mL) at room temperature were treated with EEDQ (12.83 g, 51.86 mmol). The reaction mixture was stirred under nitrogen at room temperature for 18 hours. The solvents were removed and the white solid residue was triturated with ether. The solid was collected by filtration, washed with ether and concentrated under reduced pressure to give Boc-Val-Cit-PABOH (1d). ¹H NMR (500 MHz, DMSO-d6): δ: 7.54 (d, 2H), 7.29 (d, 1H), 4.52 (s, 2H), 3.90 (d, 1H), 3.19 (m, 1H), 3.10 (m, 1H), 1.51-20.6 (m, 5H), 1.44 (s, 9H), 0.97 (d, 3H), 0.92 (d, 3H). MS (M+1): 480.0.

Boc-Val-Cit-PABC-PNP

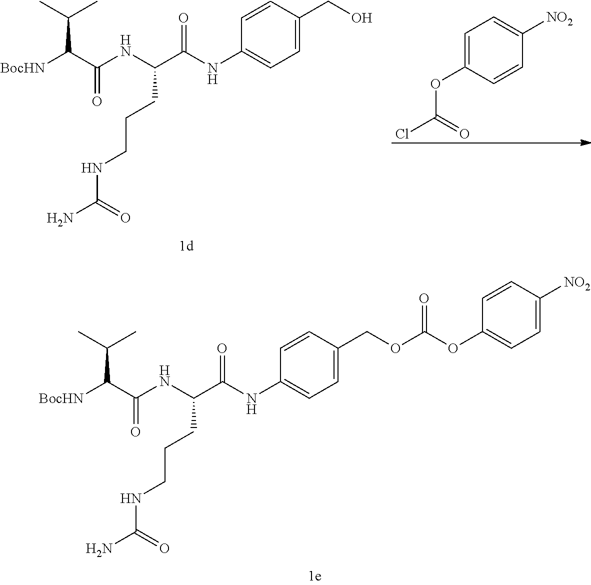

Boc-Val-Cit-PABOH (1d, 9.56 g, 19.95 mmol) under argon at room temperature was dissolved in dry pyridine (3.50 mL). The solution was cooled to 0° C., and 4-nitrophenyl chloroformate (8.69 g, 43.13 mmol) in CH₂Cl₂ was slowly added into the solution. The reaction mixture was stirred under nitrogen at room temperature for 18 hours. Remove the organic solvent, add 10% citric acid to the solution and then extracted with 10% IPA/EA. The organic layer was washed with brine, dried over MgSO4(s), and concentrated under reduced pressure to give white solid. The purified by column chromatography to give Boc-Val-Cit-PABC-PNP (le). ¹H NMR (500 MHz, DMSO-d6): δ: 8.30 (d, 2H), 7.64 (d, 2H), 7.46 (d, 2H), 7.41 (d, 2H), 5.25 (s, 2H), 4.52 (s, 1H), 3.90 (d, 1H), 3.20 (m, 1H), 3.10 (m, 1H), 1.51-20.6 (m, 5H), 1.44 (s, 9H), 0.97 (d, 3H), 0.92 (d, 3H). MS (M+1): 645.0.

Val-Cit-PABC-PNP

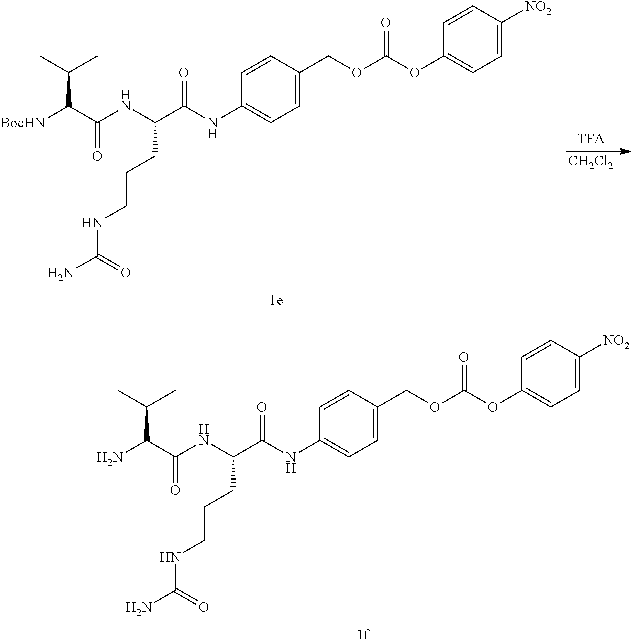

Boc-Val-Cit-PABC-PNP (le, 1.5 g) in CH₂Cl₂ (30 mL) at room temperature was treated with TFA (3.0 mL). The reaction mixture was stirred under nitrogen at room temperature for 4-5 hours. The solvents were removed and the yellow solid residue was washed with hexane. The solid was collected by filtration, washed with hexane and dried under vacuum to give Val-Cit-PABOH (le). MS (M+1): 545.

Ester-Propanoic-Val-Cit-PABC-PNP

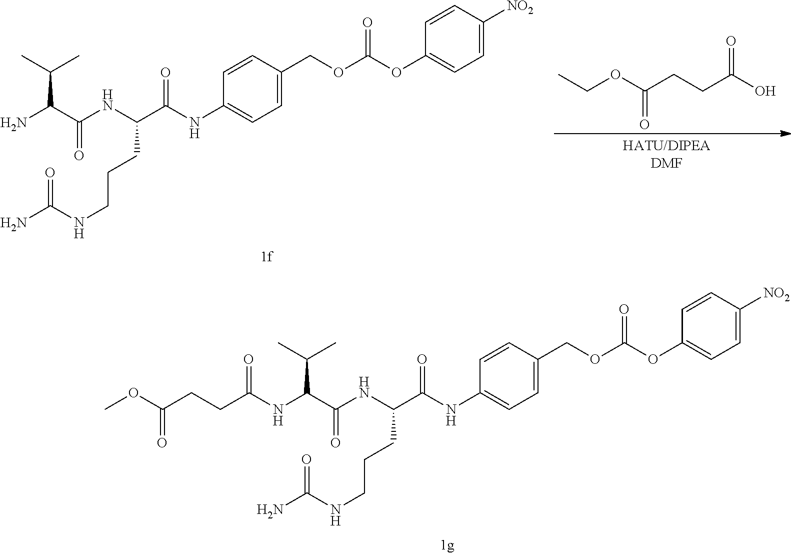

A solution of Val-Cit-PABOH (if, 544 mg, 1.0 mmol), 3-(ethoxycarbonyl) propanoic acid (292 mg, 2.0 mmol), DIPEA (0.387 g, 3.0 mmol) and HATU (0.76 g, 2.0 mmol) in DMF (10 mL) was stirred under nitrogen at room temperature for 16 hours. The reaction was quenched with water and extracted with CH₂Cl₂. The organic layer was washed with brine, dried over MgSO4(s), and concentrated under reduced pressure to give yellow solid. The yellow solid was purified by column chromatography to give Ester-Propanoic-Val-Cit-PABC-PNP (1g). 1H NMR (500 MHz, CDCl3/CD3OD): δ: 0.99 (m, 6H), 1.64 (m, 2H), 1.78 (m, 1H), 1.81 (m, 1H), 2.16 (m, 1H), 2.50-2.70 (m, 4H), 3.12 (m, 1H), 3.62 (s, 3H), 4.19 (m, 1H), 4.51 (m, 2H), 5.29 (s, 2H), 7.40-7.55 (m, 4H), 7.75 (d, 2H), 8.34 (d, 2H), 8.46 (t, 1H), 8.76 (t, 1H). MS (M+1): 659.

Ester-Propanoic-Val-Cit-PABC-Biotin

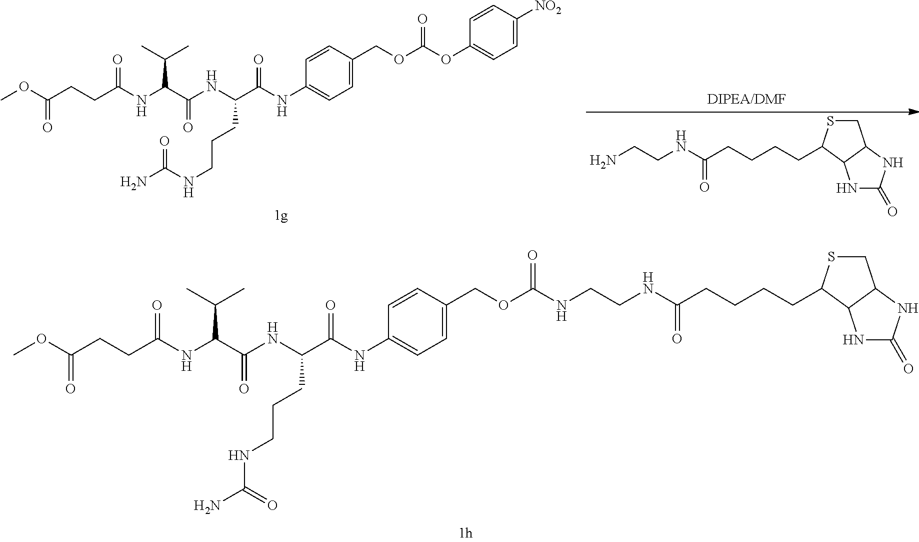

Ester-MC-Val-Cit-PABC-PNP (1g, 143 mg, 0.217 mmol) and Amino-Biotin (67 mg, 0.234 mmol) in DMF (2 mL) at room temperature were treated with DIPEA (60.6 mg). The reaction mixture was stirred under nitrogen at room temperature for 16 hours. The reaction was quenched with water and extracted with CH₂Cl₂. The organic layer was washed with brine, dried over MgSO4(s), and concentrated under reduced pressure. The residue was purified by column chromatography to give Ester-Propanoic-Val-Cit-PABC-Biotin (1h). ¹H NMR (500 MHz, DMSO-d6): δ: 0.83 (m, 6H), 1.40-1.60 (m, 11H), 2.00-2.10 (m, 2H), 2.55 (m, 1H), 2.70-3.30 (m, 7H), 3.55 (s, 3H), 4.10-4.46 (m, 4H), 4.93 (s, 2H), 5.41 (s, 2H), 5.97 (s, 1H), 6.35-6.44 (s, 2H), 7.22 (d, 1H), 7.27 (m, 2H), 7.60 (m, 2H), 7.82 (s, 1H), 7.97 (d, 1H), 8.09 (d, 1H), 9.94 (s, 1H). MS (M+1): 806.

Hydrazide-Propanoic-Val-Cit-PABC-Biotin

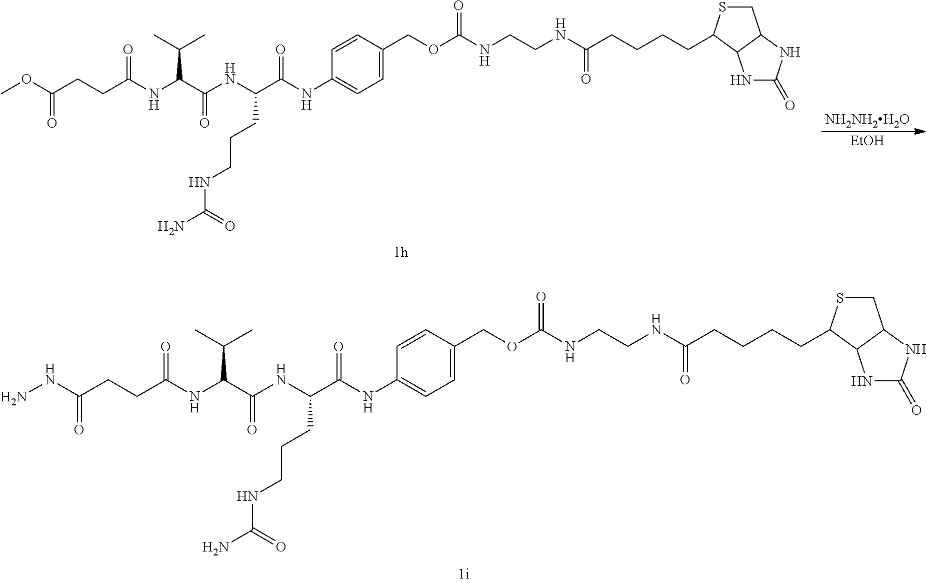

A solution of Ester-Propanoic-Val-Cit-PABC-Biotin (1h, 80.5 mg) and NH₂NH₂.H₂O (1.0 mL) in EtOH (5.0 mL) was refluxed under nitrogen for 16 hours. The mixture was cooled to room temperature and quenched with water. The solid was collected by filtration, washed with CH₂Cl₂ and MeOH to give Hydrazide-Propanoic-Val-Cit-PABC-Biotin (1i). ¹H NMR (500 MHz, DMSO-d6): δ: 0.83 (m, 6H), 1.40-1.60 (m, 11H), 2.00-2.10 (m, 2H), 2.55 (m, 1H), 2.70-3.30 (m, 7H), 3.55 (s, 3H), 4.10-4.46 (m, 4H), 4.93 (s, 2H), 5.41 (s, 2H), 5.97 (s, 1H), 6.35-6.44 (s, 2H), 7.22 (d, 1H), 7.27 (m, 2H), 7.60 (m, 2H), 7.82 (s, 1H), 7.97 (d, 1H), 8.09 (d, 1H), 9.94 (s, 1H). MS (M+1): 806.

### Example 2

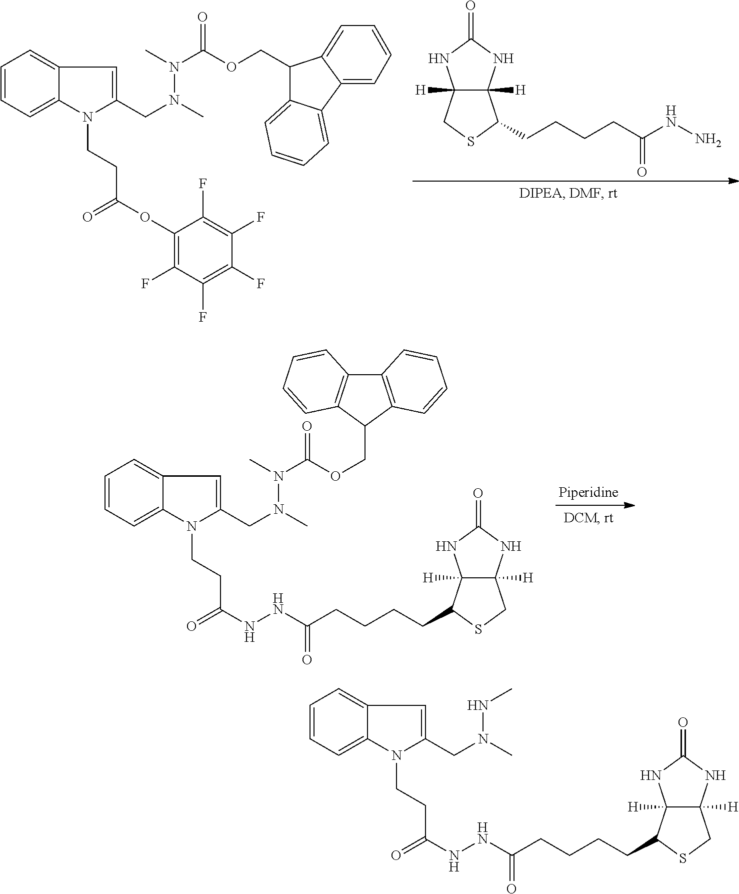

(9H-fluoren-9-yl)methyl 1,2-dimethyl-2-((1-(3-oxo-3-(2-(5-((3aS,4R,6aR)-2-oxohexahydro-1H-thieno[3,4-d]imidazol-4-yl)pentanoyl)hydrazinyl)propyl)-1H-indol-2-yl)methyl)hydrazine-1-carboxylate

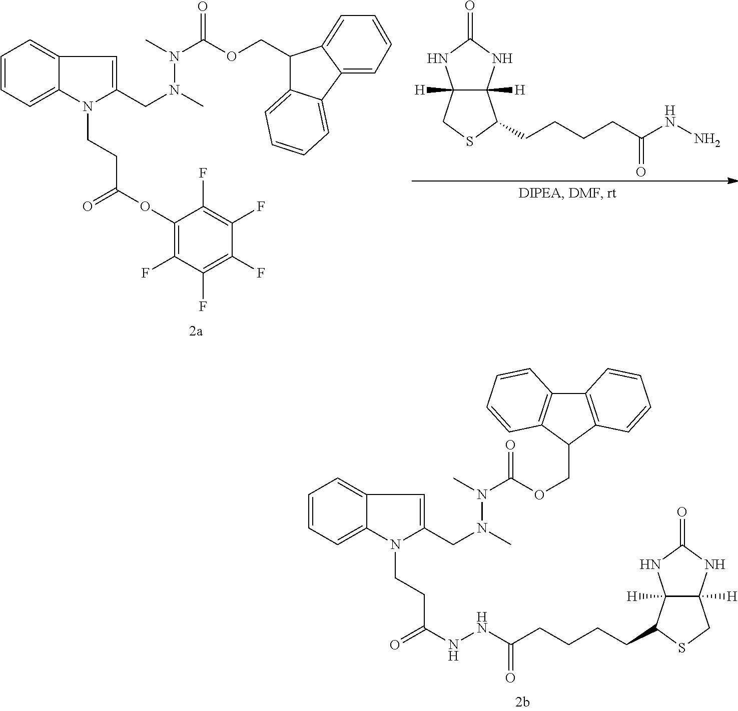

A solution of 5-((3aS,4S,6aR)-2-oxohexahydro-1H-thieno[3,4-d]imidazol-4-yl)pentanehydrazide (0.10 g, 0.39 mmol), (9H-fluoren-9-yl)methyl 1,2-dimethyl-2-((1-(3-oxo-3-(perfluorophenoxy)propyl)-1H-indol-2-yl)methyl)hydrazine-1-carboxylate (0.28 g, 0.43 mmol) and DIPEA (0.14 g, 1.04 mmol) in DMF (1.94 mL) was stirred in room temperature for 19 h. The mixture was concentrated under reduced pressure, and then was purified by C18 silica gel chromatography (0-10% MeOH in dichloromethane) to give (9H-fluoren-9-yl)methyl 1,2-dimethyl-2-((1-(3-oxo-3-(2-(5-((3aS,4R,6aR)-2-oxohexahydro-1H-thieno [3,4-d]imidazol-4-yl)pentanoyl)hydrazinyl)propyl)-1H-indol-2-yl)methyl)hydrazine-1-carboxylate (2a) (0.18 g). ESI-MS: m/z 724 (M+H)+.

### Synthesis of N′-(3-(2-((1,2-dimethylhydrazinyl)methyl)-1H-indol-1-yl) propanoyl)-5-((3aS,4R,6aR)-2-oxohexahydro-1H-thieno [3,4-d]imidazol-4-yl)pentanehydrazide

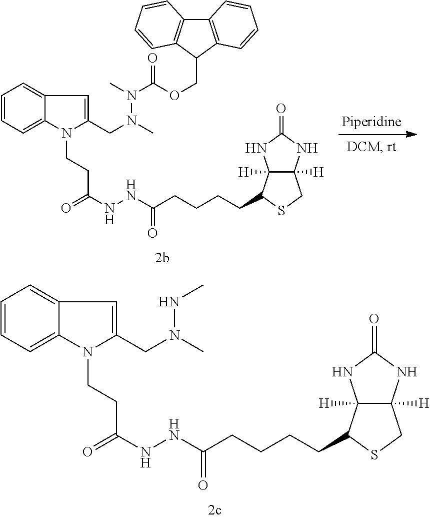

A solution of (9H-fluoren-9-yl)methyl 1,2-dimethyl-2-((1-(3-oxo-3-(2-(5-((3aS,4R,6aR)-2-oxohexahydro-1H-thieno [3,4-d]imidazol-4-yl)pentanoyl)hydrazinyl)propyl)-1H-indol-2-yl)methyl)hydrazine-1-carboxylate (0.41 g, 0.57 mmol) and piperidine (0.11 ml, 1.33 mmol) in dichloromethane (1.14 mL) was stirred in room temperature for 17 h. The mixture was concentrated under reduced pressure, and then was purified by C18 silica gel chromatography (0-10% MeOH in dichloromethane) to give N′-(3-(2-((1,2-dimethylhydrazinyl)methyl)-1H-indol-1-yl)propanoyl)-5-((3aS,4R,6aR)-2-oxohexahydro-1H-thieno[3,4-d]imidazol-4-yl)pentanehydrazide (2c) (0.16 g). ESI-MS: m/z 502 (M+H)+.

### Example 3

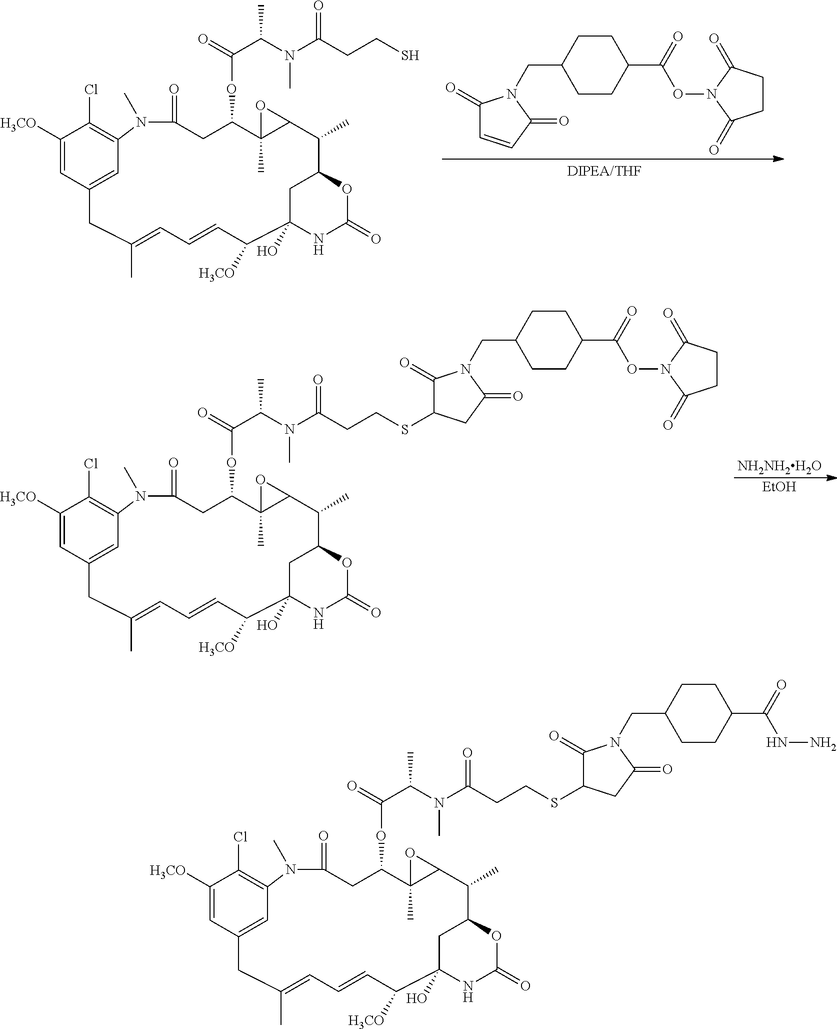

SMCC-DM1

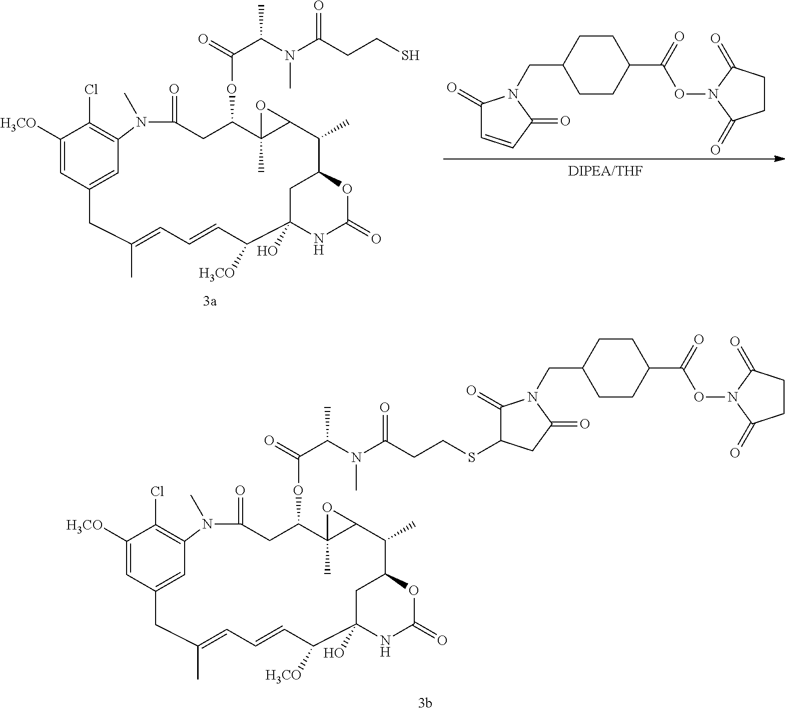 [SMILES: COc1cc2cc(c1Cl)N(C)C(=O)C[C@H](OC(=O)[C@H](C)N(C)C(=O)CCS)[C@@]1(C)OC1[C@H](C)[C@@H]1C[C@@](O)(NC(=O)O1)[C@H](OC)/C=C/C=C(\C)C2 ; O=C(ON1C(=O)CCC1=O)C1CCC(CN2C(=O)C=CC2=O)CC1 ; COc1cc2cc(c1Cl)N(C)C(=O)C[C@H](OC(=O)[C@H](C)N(C)C(=O)CCSC1CC(=O)N(CC3CCC(C(=O)ON4C(=O)CCC4=O)CC3)C1=O)[C@@]1(C)OC1[C@H](C)[C@@H]1C[C@@](O)(NC(=O)O1)[C@H](OC)/C=C/C=C(\C)C2]

DM1 (87 mg, 0.117 mmol) and SMCC (118 mg, 0.354 mmol) in THF (2.0 mL) at room temperature were treated with DIPEA (45.7 mg, 0.354 mmol). The reaction mixture was stirred under nitrogen at room temperature for 18 hours. The reaction was quenched with water and extracted with CH₂Cl₂. The organic layer was washed with brine, dried over MgSO4(s), and concentrated under reduced pressure to give yellow solid. The yellow solid was purified by column chromatography to give SMCC-DM1 (3a). ¹H NMR (500 MHz, CDCl₃): δ: 0.80 (m, 3H), 1.02 (m, 3H), 1.20-2.00 (m, 25H), 2.0-14.00 (m, 36H), 4.12 (s, 3H), 4.27 (m, 1H), 4.78 (d, 1H), 5.30 (s, 1H), 5.39 (m, 1H), 5.7 (m, 1H), 6.26 (s, 1H), 6.43 (m, 1H), 6.60-6.71 (m, 2H), 6.83 (d, 1H). MS(M+1): 1073.

Hydrazide-SMCC-DM1

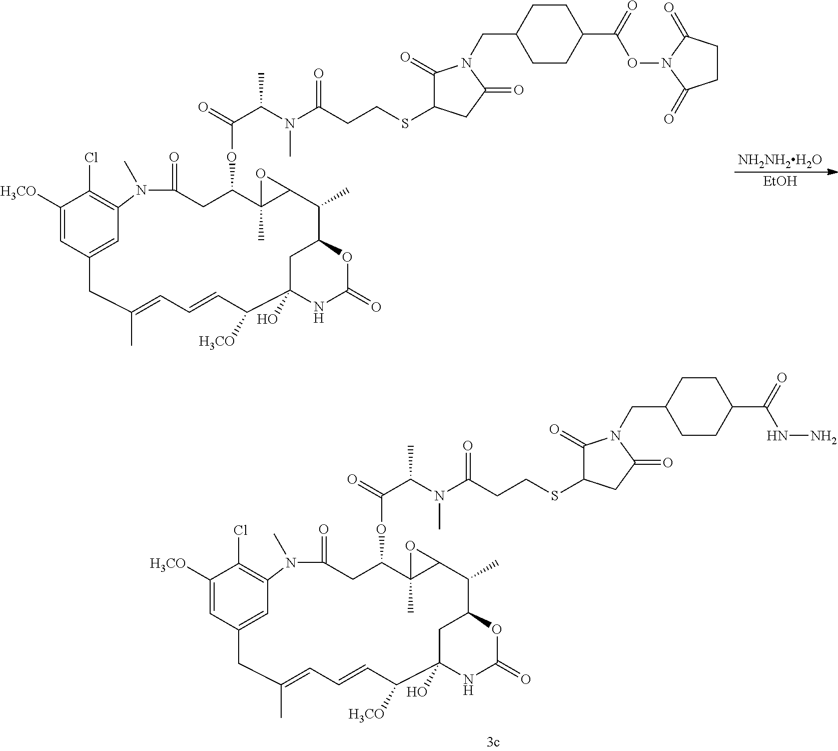 [SMILES: "EtOH" ; COc1cc2cc(c1Cl)N(C)C(=O)C[C@H](OC(=O)[C@H](C)N(C)C(=O)CCSC1CC(=O)N(CC3CCC(C(=O)ON4C(=O)CCC4=O)CC3)C1=O)[C@@]1(C)OC1[C@H](C)[C@@H]1C[C@@](O)(NC(=O)O1)[C@H](OC)/C=C/C=C(\C)C2 ; "NH2NH2·H2O" ; COc1cc2cc(c1Cl)N(C)C(=O)C[C@H](OC(=O)[C@H](C)N(C)C(=O)CCSC1CC(=O)N(CC3CCC(C(=O)NN)CC3)C1=O)[C@@]1(C)OC1[C@H](C)[C@@H]1C[C@@](O)(NC(=O)O1)[C@H](OC)/C=C/C=C(\C)C2]

A solution of SMCC-DM1 (107.3 mg, 0.1 mmol) and NH₂NH₂.H₂O (1.0 mL) in EtOH (3.0 mL) was refluxed under nitrogen for 2 hours. The reaction was quenched with water and extracted with CH₂Cl₂. The organic layer was washed with brine, dried over MgSO4(s), and concentrated under reduced pressure. The residue was purified by column chromatography to give [symbol] Hydrazide-SMCC-DM1(3b). ¹H NMR (500 MHz, CDCl₃): δ: 0.80 (m, 3H), 1.23 (m, 2H), 1.20-2.00 (m, 25H), 2.0-14.00 (m, 36H), 3.99 (s, 3H), 4.27 (m, 1H), 4.79 (d, 1H), 5.34 (s, 1H), 5.65 (m, 1H), 6.27 (d, 1H), 6.44 (m, 1H), 6.63 (m, 2H), 6.93 (d, 1H), 7.26 (s, 1H). MS (M+1): 990.

Preparation of Herceptin-GlcNAc-Fuc

Herceptin (10 mg in 500 μL), 10*GlycoBuffer (100 μL), EndoS (10 μL) and double distilled water (390 μL) were placed together in a vial and stirred in 37° C. for 16 hours. Then the solution was added with 9.0 ml PBS and 1.0 ml protein A beads in centrifuge tube and rotate at 25° C. for 1.0 hour. Then the reaction mixture was centrifuged under 4000 rpm at 4° C. for 5.0 minutes, The resin column was washed by 10 ml PBS. The antibodies are eluted with 100 mM glycine and immediately neutralized to pH 8.0 Tris in the tube.

Preparation of Anti-Her2 mAb-GlcNAc-Gal

The anti-Her2 mAb-GlcNAc-Gal is prepared by chemical transfection or electroporation of anti Her2 antibody expression vector into Endo S-transfected-CHO cells or 293HEK cells. Briefly, for production of anti-Her2 mAb-GlcNAc-Gal, the host cells were engineered by transfected with Endo S expression vector which can cleavage β 1-4-linkage glycosidic bond between to two GlcNAc and leave GlcNAc-moiety on Asn297 residue of anti Her2 antibody. However, EndoS-expressing CHO cells or F293 cells contained three sugar combinations after introducing anti Her2 expression vector, i.e., mono-GlcNAc (about 50%-60%), GlcNAc linked with galactose (GlcNAc-Gal) (about 40%-50%), and GlcNAc linked with galactose followed with Sialic acid (GlcNAc-Gal-Sia) (less than 10%). For preparation of anti-Her2 mAb-GlcNAc-Gal, the cultured medium containing three sugar combinations anti Her2 mAb was purified by protein A affinity chromatographic procedure. The extra sailic acids of GlcNAc linked with galactose followed with Sialic acid (GlcNAc-Gal-Sia) was trimmed by Neurominidase treatment. The final products of anti Her2 antibodies including mono-GlcNAc (about 33%), GlcNAc linked with galactose (GlcNAc-Gal) (about 67%) can be obtained by further protein A affinity chromatographic procedure.

Preparation of Anti-Her2 mAb-GlcNAc

For preparation of anti-Her2 mAb-GlcNAc monoclonal clonal antibody, post translational glycosylation-restricted cell lines were developed. The glycosylation-restricted cell lines is prepared by chemical transfection or electroporation of Endo S-transfected-CHO cells or 293HEK cells. However, EndoS-expressing CHO cells or F293 cells contained three sugar combinations after introducing anti Her2 expression vector, i.e., mono-GlcNAc (about 50%-60%), GlcNAc linked with galactose (GlcNAc-Gal) (about 40%-50%), and GlcNAc linked with galactose followed with Sialic acid (GlcNAc-Gal-Sia) (less than 10%). For production of anti-Her2 mAb-GlcNAc, the cultured medium of anti Her2 mAb-transfected Endo S expressing CHO cells was purified by protein A column. The three sugar combinations of anti Her2 mAb can be purified from cultured supernatants of Endo S-expressing CHO cells or 293 HEK cell stable pools by protein A affinity chromatographic procedure. To obtain anti-Her2 mAb-GlcNAc monoclonal antibody, the extra sailic acids and galactose of the anti-Her2 mAb-GlcNAc-Gal-sailic acid can be removed by further Neuraminidase and galactosidase treatment to obtain pure anti-Her2 mAb-GlcNAc.

Oxidation Through NaIO₄

Different glycan types of monoclonal antibodies in pH 6.0, 10 mM Sodium phosphate Buffer/150 mM NaCl were placed in brown vials (protect from light) and NaIO₄ (with different equivalent) were added and stirred under argon(at different temperature) for 0.5-2.0 hours. The solution was desalted and concentrated through Amicon Ultra-15 centrifugal filter device with 30 kDa NMWL in pH 6.0, 1.0M Sodium acetate Buffer. Then dilute the mAb to 2.0 mg/mL with pH 6.0, 1.0M Sodium acetate Buffer.

Oxidation Through Galactose Oxidase

Different glycan types of monoclonal antibodies in 1.0 M Potassium phosphate buffer (pH 6.0), were placed in brown vials (protect from light) and Galactose oxidase (sigma or Worthington) was added and stirred under argon(at different temperature) for 16-72 hours.

Conjugation Protocol

Different glycan types of monoclonal antibodies after oxidation were placed in brown vials (protect from light), linkers and payload (biotin or DM1) with different conjugation moieties (hydrazide or HIPS) were added and stirred under argon (at different temperature) for 16-72 hours. The solution was desalted and concentrated through Amicon Ultra-15 centrifugal filter device with 30 kDa NMWL in pH 7.4 PBS buffer.

ELISA Assay for Biotinylation Assay

IgG in Coating buffer at concentration of 1 μg/ml were coated plate with 100 μL/well. The plates were sealed and incubated at 4° C. overnight. Aspirate wells and wash 3 times with 300 μL/well PBST (0.05% Tween 20). Block well with 300 μL/well of PBS-5% skim milk incubated at 37° C. for 1 hour. Aspirate wells and wash 3 times with 300 μL/well. Add 100 ng ADC sample per 100 μL/well diluted with PBS and incubated at 37° C. for 1 hour. Aspirate wells and wash 3 times with 300 μL/well PBST(0.05% Tween 20). Add 100 μL/well Streptavidin (1:10000) and incubated at 37° C. for 1 hour. Aspirate wells and wash 3 times with 300 μL/well PBST (0.05% Tween 20). Add 100 μL/well of TMB at 37° C. for 10 minutes. The color development can be stopped by adding 1004, of 1N HCl. And read plates by measure absorbance of 450-650 nm using the ELISA reader.

ELISA Assay for Binding Affinity

Her2 in Coating buffer at concentration of 1 μg/ml were coated plate with 100 μL/well. The plates were sealed and incubated at 4° C. overnight. Aspirate wells and wash 3 times with 300 μL/well PBST (0.05% Tween 20). Block well with 300 μL/well of PBS-5% skim milk incubated at 37° C. for 1 hour. Aspirate wells and wash 3 times with 300 μL/well. Add 100 ng ADC sample per 100 μL/well diluted with PBS-1% BSA and incubated at 37° C. for 1 hour. Aspirate wells and wash 3 times with 300 μL/well PBST(0.05% Tween 20). Add 100 μL/well anti-human Kappa light chains (1:10000) and incubated at 37° C. for 1 hour. Aspirate wells and wash 3 times with 300 μL/well PBST (0.05% Tween 20). Add 100 μL/well of TMB at 37° C. for 10 minutes. The color development can be stopped by adding 1004, of 1N HCl. And read plates by measure absorbance of 450-650 nm using the ELISA reader.

LC/MS Detection of ADC

Liquid chromatography-time-of-flight mass spectrometry (LC-TOF MS) analysis is a key tool for determining the exact molecular weights of the glycoforms- and any heterogeneity within the monoclonal antibody preparations. In order to characterize the prepared mAb, the TOF MS spectra and resulting reconstructed mass graphs of this denatured and reduced mAb for the light and heavy chains, were obtained by using liquid chromatography-mass spectrometry analysis, respectively. Heavy chain on the other hand gives a much more heterogeneous TOF MS spectrum because of the glycosylation that commonly takes place on this part of the antibody. The heavy chain shows multiple peaks, attributable to the heterogeneity in glycosylation at Asn297. The reconstructed mass graph confirms that the major peaks for the heavy chain are a series of proteins with different sizes of glycans attached to them. Mass differences of ˜162 Da and 203 Da are indicative of different sugar moieties on the glycan structure.

1. A method for specific linkage to a glycoprotein, comprising:
obtaining a glycoprotein having a monoglycan or diglycan attached thereto;
producing a reactive functional group on a sugar unit on the glycoprotein; and
coupling a linker or a payload to the reactive functional group on the glycoprotein.

2. The method according to claim 1, wherein the glycoprotein is one selected from the group consisting of mono-glycan(GlcNAc), diglycan (GlcNAc-Fuc), and diglycan (GlcNAc-Gal) and the linker comprise one selected from the group consisting of a hydrazide moiety, a hydrazino-Pictet-Spengler ligation moiety, an amine moiety, an oxazoline moiety, a methylhydrazine, and an N-methyl hydroxylamine.

3. The method according to claim 2, wherein the producing a reactive functional group is by NaIO₄ oxidation.

4. The method according to claim 2, wherein the producing a reactive functional group is by galactose oxidase oxidation.

5. The method according to claim 2, wherein the glycoprotein is mono-glycan(GlcNAc) and the linker comprise a functional group selected from the group consisting of a hydrazide moiety, a hydrazino-Pictet-Spengler ligation moiety, an amine moiety, an oxazoline moiety, a methylhydrazine, and an N-methyl hydroxylamine.

6. The method according to claim 5, wherein the producing a reactive functional group is by NaIO₄ oxidation.

7. The method according to claim 5, wherein the producing a reactive functional group is by galactose oxidase oxidation.

8. The method according to claim 1, further comprising coupling a payload to the linker after the coupling of the linker.

9. The method according to claim 1, wherein the payload is a therapeutic agent, a cytotoxic agent, or an imaging agent.

10. The method according to claim 8, wherein the payload is a therapeutic agent, a cytotoxic agent, or an imaging agent.
